(19) United States
(12) Patent Application Publication
(10) Pub. No.: US 2006/0211970 A1
(43) Pub. Date: Sep. 21, 2006

(54) Releasable application systems and releasable medical device systems

(76) Inventor: Frank J. Sciulli, Crafton, PA (US)

Correspondence Address: GREGORY L BRADLEY;MEDRAD INC, ONE MEDRAD DRIVE, INDIANOLA, PA, 15051, US

(21) Appl. No.: 11/082,209

(22) Filed: Mar. 16, 2005

(51) Int. Cl. A61F 13/00; A61F 15/00

(52) U.S. Cl. 602/41; 602/57; 602/58

(57) ABSTRACT

A medical sensor system for attachment to a patient includes a patient structure which includes a patient backing layer having a first major surface and a second major surface. A first adhesive layer coves at least a portion of the first major surface. The first adhesive layer is adapted to adhere to the patient. A second adhesive layer covers at least a portion of the second major surface. The system further includes an intermediate layer attached (for example, adhered) to the second adhesive layer and a sensor structure removably attached (for example, adhered) to the intermediate layer. The sensor structure can, for example, include a sensor backing layer having a third major surface and a fourth major surface. A third adhesive layer can cover at least a portion of the third major surface. The third adhesive layer can be adapted to adhere to the intermediate layer. A fourth adhesive layer covers at least a portion of the fourth major surface. The sensor system further includes a sensor attachable to the fourth adhesive layer of the sensor backing layer. In one embodiment, the patient backing layer and the first adhesive layer are stretchable together to effect debonding of the first adhesive layer from the patient. Likewise, the sensor backing layer and the third adhesive layer can be stretchable together to effect debonding of the third adhesive layer from the intermediate layer.

### BACKGROUND OF THE INVENTION

The present invention relates generally to releasable application systems and, particularly, to releasable application systems for use in the medical field to releasably attach a medical device such as a sensor to a patient.

Adhesive sheets or adhesive materials such as adhesive tapes are widely used in the medical field. For example, adhesive tapes are used to maintain certain medical devices (for example, catheters, sensors etc.) in close proximity to the body of a patient. In many instances, adhesive tape is simply pulled over a device and adhered to the patient to maintain the device in connection with the patient. Likewise, many bandages or other tissue coverings include an adhesive material.

Often it is difficult to remove such adhesive tapes, strips or sheets from connection with the patient. Moreover, removal of such adhesive materials from the patient is often a painful experience for the patient. U.S. Pat. No. 4,024,312, the disclosure of which is incorporated herein by reference, describes a highly conformable adhesive tape which includes a highly extensible and elastic backing film laminated with an adhesive layer to form the tape. The adhesive is coated on at least one of the backing film surfaces and is a normally tacky and pressure-sensitive elastomeric adhesive. The tape of U.S. Pat. No. 4,024,312 is easily stretchable and can be removed easily from an application surface by stretching the tape lengthwise in a direction substantially parallel to the surface. The adhesive tape is described for use in medical applications where painless removal is desirable such as in so-called “ouchless” bandages.

The type of removable adhesive tape laminate (which can be a single-sided or a double-sided adhesive tape) used in U.S. Pat. No. 4,024,312 that is stretchable to accomplish removal is sometimes referred to as a stretch release adhesive tape. Such an adhesive tape can, for example, include a conventional pressure-sensitive adhesive on one or both major surfaces thereof, and a tape backing formed from a highly extensible material. Such adhesive materials can also be formed from a solid adhesive material that itself provides one or two adhesive surfaces and is sufficiently extensible. When the backing or solid adhesive layer is stretched subsequent to adhering the tape on one or both sides, the adhesive stretches and undergoes a generally clean interfacial debonding from the substrate or substrates to which it is adhered. Sometimes, a pull tab portion at the end of the strip is provided to facilitate the stretching and debonding. Stretch release adhesive tapes are described, for example, in U.S. Pat. Nos. 5,409,189, 5,516,581, 5,672,402, 5,989,708 and 6,527,900, the disclosures of which are incorporated herein by reference. A stretch release tape laminate structure having an internally separable layer is described in U.S. Pat. No. 6,004,642, the disclosure of which is incorporated herein by reference. Stretch release adhesive tape laminates including a separable fastener are disclosed in U.S. Pat. Nos. 6,572,945 and 6,692,807, the disclosures of which are incorporated herein by reference. Many of the stretch release adhesive tapes can, for example, include conventional pressure sensitive adhesives that can provide a strong bond against shear, peel and lift-off forces.

It is desirable to develop devices, systems and methods whereby a medical device can be releasably or removably attached to a patient.

### SUMMARY OF THE INVENTION

In one aspect, the present invention provides a medical system for attachment to a patient. The medical system includes a patient structure having a patient structure backing layer including a first major surface and a second major surface. A first patient structure adhesive layer covers at least a portion of the first major surface. The first patient structure adhesive layer is adapted to adhere to the patient. The medical system further includes a device structure including a device structure backing layer including a third major surface and a fourth major surface. The device structure also includes a first device structure adhesive layer covering at least a portion of the fourth major surface. The third major surface is in removable connection with the patient structure.

In one embodiment, the patient structure is adapted to remain adhered to the patient after the device backing layer is removed from connection with the patient structure. The patient structure can, for example, form at least part of a bandage system.

In one embodiment, the medical system further includes an intermediate layer. The patient structure can further include a second patient structure adhesive layer covering at least a portion of the second major surface. The second patient structure adhesive is adhered to the intermediate layer. The device structure can further include a second device structure adhesive layer covering at least a portion of the third major surface of the device structure backing layer. The second device structure adhesive layer is adhered to the intermediate layer.

The device backing layer and the second device structure adhesive layer can be stretchable together to effect debonding of the second device structure adhesive layer from the intermediate layer. Similarly, the patient structure backing layer and the first patient structure adhesive layer can be stretchable together to effect debonding of the first patient structure adhesive layer from the patient.

The medical system can further include a medical device attachable to the first device structure adhesive layer. The medical device can, for example, be a sensor. The sensor can, for example, be an extravasation sensor. In one embodiment, the extravasation sensor includes at least one transmitter for transmitting electromagnetic energy in the range of 300 MHz to 30 Ghz into tissue of the patient and at least one receiver to receive a resultant signal. In one such embodiment, the at least one transmitter is adapted to transmit electromagnetic energy having a frequency greater than 1.5 GHz.

In another aspect, the present invention provides a system for removable attachment of a medical device to a patient. The system includes a patient structure including a first patient structure adhesive layer adapted to adhere to a patient and a cover layer in connection with the first patient structure adhesive layer. A medical device structure is in removable connection with the cover layer of the patient structure. The medical device structure is removable from attachment to the cover layer of the patient structure without removing the patient structure from attachment to the patient. The medical device structure includes a first device structure adhesive layer on at least a portion of a surface opposite of a surface thereof that is in removable connection with the cover layer. The first device structure adhesive layer is adapted to attach the medical device to the medical device structure.

The patient structure can, for example, form at least part of a bandage system. In one embodiment, the system further includes a first removable protective layer in operative connection with the first patient structure adhesive layer. The first protective layer is removable to allow contact of the first patient structure adhesive layer with the patient. The system of can also include a second removable protective layer in operative connection with the first device structure adhesive layer. The second protective layer is removable to allow contact of the first device structure adhesive layer with the medical device.

In a further aspect, the present invention provides a method of removably attaching a medical device to a patient. The method includes the step of attaching an attachment system to the patient. The attachment system includes a patient structure including a first adhesive layer adapted to adhere to the patient and a cover layer in connection with the first adhesive layer. The attachment system further includes a medical device structure removably attached to the cover layer of the patient structure. The medical device structure includes a device structure adhesive on a surface thereof opposite of a surface thereof in removable connection with the cover layer. The device structure adhesive is adapted to attach the medical device to the medical device structure. The method can further include removing the medical device structure from attachment to the cover layer of the patient structure without removing the patient structure from attachment to the patient. The method can also include the step of attaching the medical device to the device structure adhesive.

In one embodiment, the medical device is an extravasation sensor. The extravasation sensor can, for example, include at least one transmitter for transmitting electromagnetic energy in the range of 300 MHz to 10 Ghz into tissue of the patient and at least one receiver to receive a resultant signal.

In another aspect, the present invention provides a medical sensor system for attachment to a patient. The medical sensor system includes a patient structure which includes a patient backing layer having a first major surface and a second major surface. A first adhesive layer coves at least a portion of the first major surface. The first adhesive layer is adapted to adhere to the patient. A second adhesive layer covers at least a portion of the second major surface. The system further includes an intermediate layer attached (for example, adhered) to the second adhesive layer and a sensor structure removably attached (for example, adhered) to the intermediate layer. The sensor structure can, for example, include a sensor backing layer having a third major surface and a fourth major surface. A third adhesive layer can cover at least a portion of the third major surface. The third adhesive layer can be adapted to adhere to the intermediate layer. A fourth adhesive layer covers at least a portion of the fourth major surface. The sensor system further includes a sensor attachable to the fourth adhesive layer of the sensor backing layer. In one embodiment, the patient backing layer and the first adhesive layer are stretchable together to effect debonding of the first adhesive layer from the patient. Likewise, the sensor backing layer and the third adhesive layer can be stretchable together to effect debonding of the third adhesive layer from the intermediate layer.

In one embodiment, the patient structure remains adhered to the patient after the sensor backing layer is stretched to remove the sensor form connection with the intermediate layer. The patient structure forms at least part of a tissue covering (for example, a bandage) system.

In another aspect, the present invention provides a medical device application system for attachment of a medical device to a patient. The application system includes a patient structure including a patient backing layer having a first major surface and a second major surface. A first adhesive layer covers at least a portion of the first major surface and is adapted to adhere to the patient. A second adhesive layer covers at least a portion of the second major surface. An intermediate layer is adhered to the second adhesive layer. The application system further includes a device structure including a device backing layer having a third major surface and a fourth major surface. A third adhesive layer covers at least a portion of the third major surface and is adapted to adhere to the intermediate layer. A fourth adhesive layer covers at least a portion of the fourth major surface and is adapted to adhere to the medical device. The device backing layer and the third adhesive layer are stretchable together to effect debonding of the third adhesive layer from the intermediate layer.

In still a further aspect, the present invention provides a medical sensor system for attachment to a patient to detect extravasation during an injection. The system includes a patient structure having a an adhesive layer adapted to adhere to a patients skin and cover layer. The system also includes a stretchable backing layer comprising a first major surface and a second major surface. A first adhesive layer covers at least a portion of the first major surface and is adapted to adhere to the cover layer of the bandage. A second adhesive layer covers at least a portion of the second major surface. The system further includes a sensor attachable to the second adhesive layer of the stretchable backing layer. As described above, the stretchable backing layer and the first adhesive layer are preferably stretchable together to effect debonding of the first adhesive layer from the backing layer of the bandage. In one embodiment, the sensor uses microwave or other energy transmitted into the patients tissue to detect an extravasation. In that regard, the sensor can include at least one transmitter and at least one receiver.

In still another aspect, the present invention provides a medical device application system for removable attachment of a medical device to a patient. The application system includes a patient structure including an adhesive layer adapted to adhere to a patient's skin and a cover layer in connection with the adhesive layer. The application system further includes a medical device structure removably attached to the cover layer of the patient structure. The medical device structure is removable from attachment to the cover layer of the patient structure without removing the patient structure from attachment to the patient. The medical device structure includes an adhesive on a surface opposite of a surface thereof attached to the cover layer. The adhesive being is suitable to or adapted to attach the medical device to the medical device structure.

### BRIEF DESCRIPTION OF THE DRAWINGS

Other aspects of the invention and their advantages will be discerned from the following detailed description when read in connection with the accompanying drawings, in which:

FIG. 1A illustrates an embodiment of a patient structure (for example, a tissue covering or bandage) including a stretch release adhesive system in operative connection with an injection site on a patient.

FIG. 1B illustrates a top, enlarged view of the stretch release adhesive patient structure of FIG. 1A.

FIG. 1C illustrates a side, cross-sectional view of the stretch release adhesive patient structure of FIG. 1A.

FIG. 1D illustrates a side, cross-sectional view of the stretch release adhesive patient structure of FIG. 1A in which a backing layer and a patient adjacent adhesive layer are being stretched to cause debonding of the adhesive layer from the patient.

FIG. 2A illustrates a top view of the stretch release adhesive patient structure of FIG. 1A in which the stretchable backing layer is shown in dashed lines.

FIG. 2B illustrates a front view of the patient structure of FIG. 1A.

FIG. 2C illustrates a top view of the patient structure of FIG. 1A in which a tension/stretching force is being applied to an extending tab of the backing layer.

FIG. 2D illustrates a side view of the patient structure of FIG. 1A in which a tension/stretching force is being applied to an extending tab of the backing layer.

FIG. 3 illustrates an embodiment of a medical device (for example, a sensor) application system of the present invention.

FIG. 4 illustrates a side, partially cross-sectional view of the system of FIG. 3.

FIG. 5 illustrates a side, partially cross-sectional view of the system of FIG. 3, wherein the sensor has been removed from connection with the patient structure.

### DETAILED DESCRIPTION OF THE INVENTION

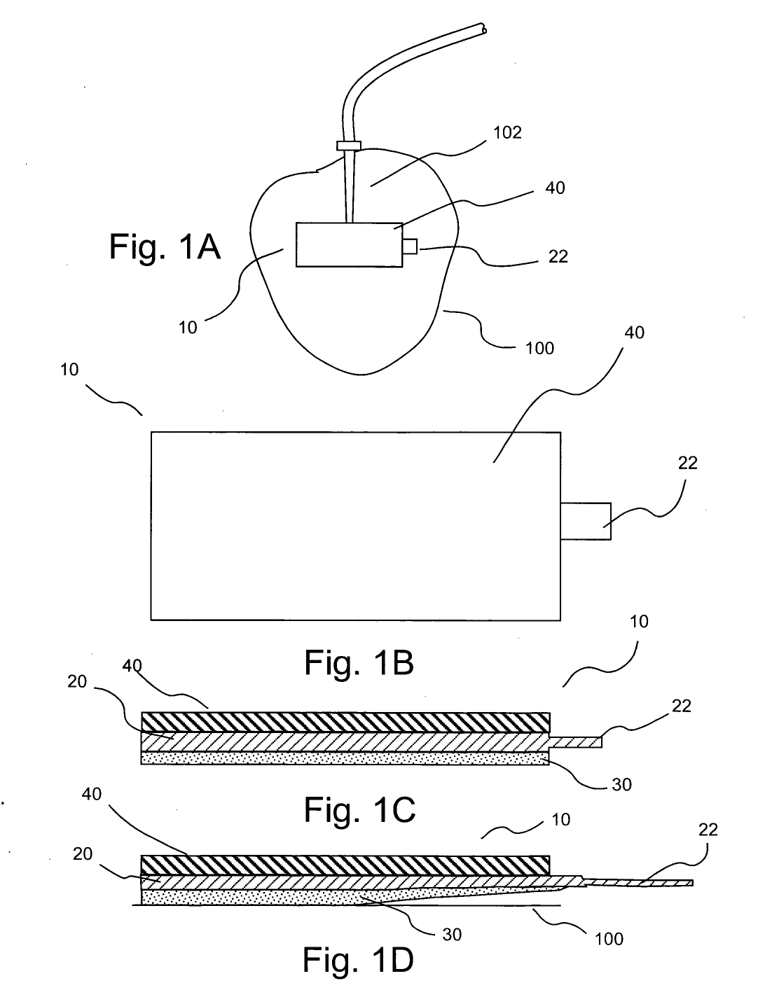

To illustrate the use of stretch release adhesives in the present invention, FIG. 1A illustrates a patient structure 10 (for example, a tissue covering or bandage) including a stretchable backing layer 20 and an adhesive layer 30. Patient structure 10 further includes a surface or protective layer 40 as known in the art which can, for example, be adhered to backing layer 20. Patient structure 10 is shown adhered to patient tissue/skin 100 in the area an injection site of a catheter 102. Materials suitable for the backing layer 20 and adhesive layer 30 are, for example, set forth in U.S. Pat. Nos. 5,516,581 and 6,572,945. As illustrated in FIGS. 1A through 1D, backing layer 220 includes a tab 22. As illustrated, for example, in FIG. 1D, a user can grasp tab 22 and apply a tension or stretching force thereto to cause stretching of backing layer 20 and adhesive layer 30 to effect debonding of adhesive layer 30 from patient 100 and removal of patient structure 10 from connection with patient 100.

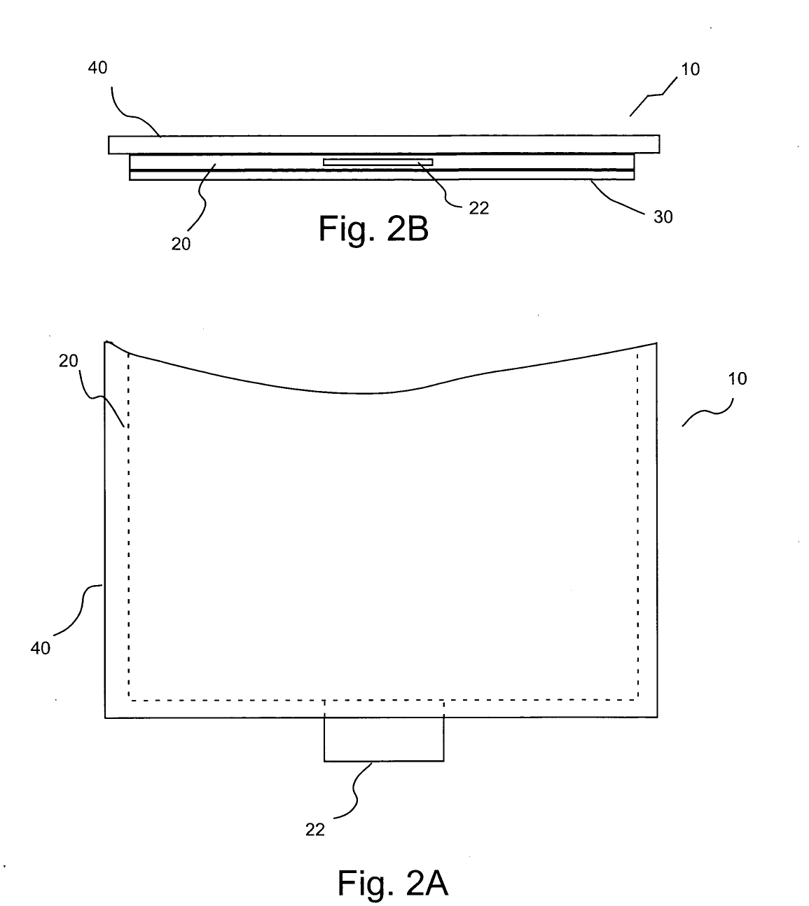

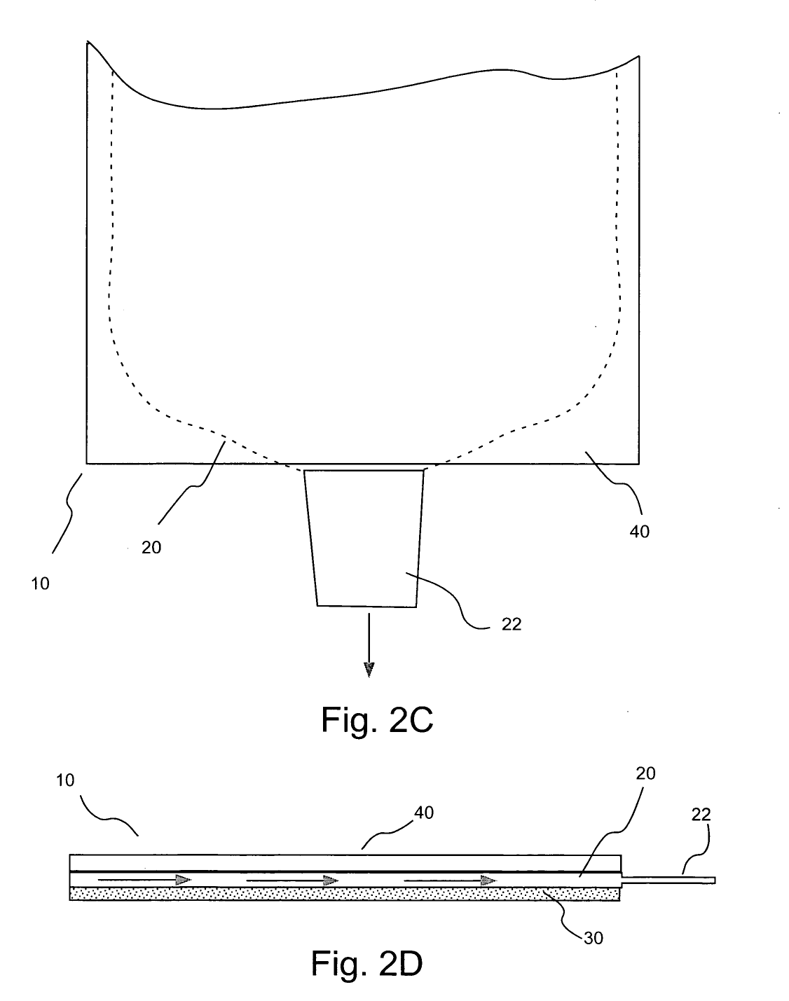

FIGS. 2A illustrates a top view of patient structure 10 wherein stretchable backing layer 20 is illustrated in dashed lines. As further illustrated in FIGS. 1C, 1D, 2C and 2D, application of force to tab 22 causes stretching/deformation of backing layer 20 and debonding of adhesive layer 30.

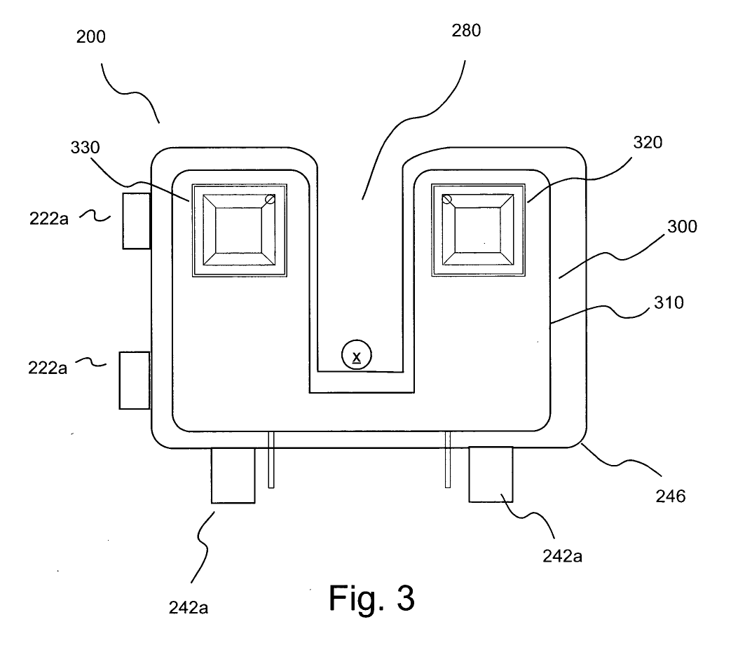

FIGS. 3-5 illustrate one embodiment of a medical device application system 200 of the present invention. Many types of medical devices, including, for example, sensors, which operate in connection with the surface of a patient's body can be used with the application systems of the present invention. For example, sensors for the detection of extravasation during an injection procedure can be used. Such sensors are disclosed, for example, in Published PCT International Application Nos. WO 03/009753 and WO 03/009752, assigned to the assignee of the present invention, the disclosures of which are incorporated herein by reference.

The application systems of the present invention allow relatively quick and simple attachment of the medical device and subsequent detachment thereof. The medical device can, for example, be reused at a later time in connection with the same patient or with a different patient. Sterilization of the medical device may or may not be required. A patient adjacent structure of the application systems of the present invention preferably remains in contact with the patient after removal of the medical device. The patient structure can, for example, act (either alone or in operative connection with another structure or structures) as a covering or bandage of an area of interest. After removal, the patient structure may constitute hazardous waste and can be disposed of in accordance with appropriate procedures.

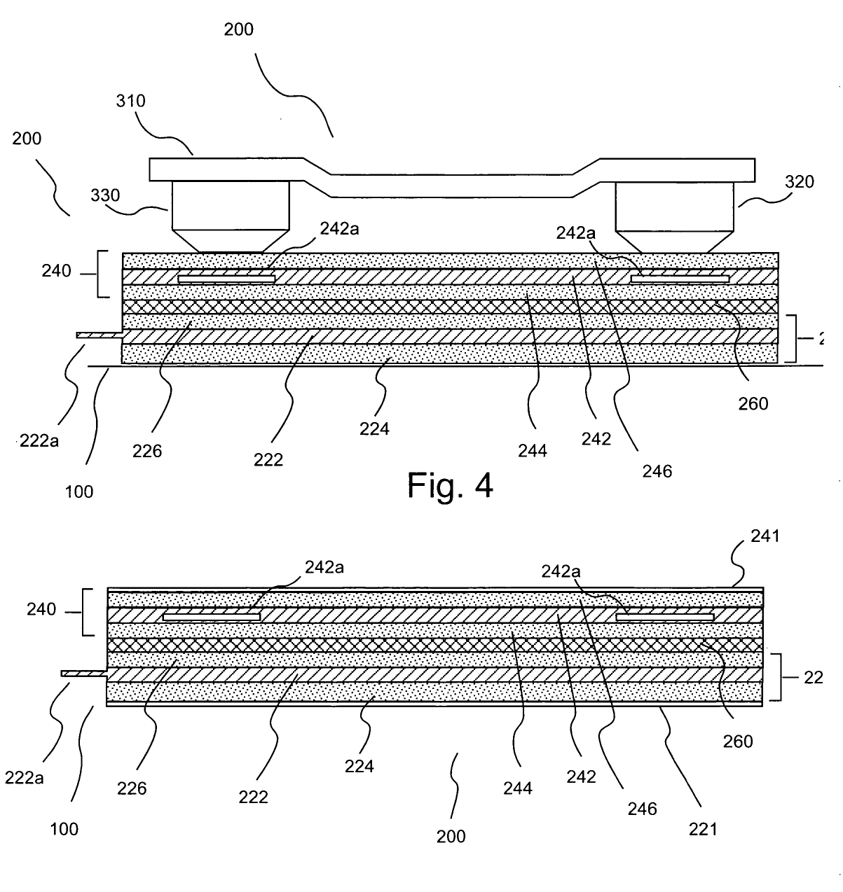

In the embodiment of FIGS. 3-5, application system 200 includes a patient structure 220 (see, for example, FIG. 4), which can, for example, be a first stretch release adhesive tape structure and a device structure 240 (see, for example, FIG. 4), which can, for example, be a second stretch release adhesive tape structure. As illustrated, for example, in FIG. 4, patient structure 220 and medical device structure 240 are connected together by an intermediate layer 260. Alternatively, one of the first or second stretch release adhesive tape structures can be replaced with a non-stretch release adhesive, for example, a pressure-sensitive adhesive or an epoxy adhesive. As shown, for example, in FIG. 4, application system 200 can be positioned between a medical device (in this embodiment, an extravasation sensor 300) and patient tissue/skin 100. As described in detail in Published PCT International Application Nos. WO 03/009753 and WO 03/009752, sensor 300 can, for example, include a base 310, at least one microwave transmitter 320 and at least one microwave receiver 330.

Sensor 300 can, for example, operate by applying electromagnetic energy (for example, in the frequency range of approximately 300 MHz to approximately 30 GHz) via transmitting antennae 320 to a volume of the body between transmitting antenna 320 and measuring a resultant or returned signal received by receiving antennae 330. A single antenna can act as both the transiting and receiving antenna. Moreover, multiple transmitting antennae and/or multiple receiving antennae can be used. The measured signal can, for example, be compared to a reference signal to determine if the fluid level in the tissue has changed (in the volume of the body or in the vicinity of the volume of the body) in a manner associated with an extravasation event. It is apparent to one skilled in the art that various other medical devices, including other sensor configurations are suitable for use with the attachment devices of the present invention.

The first stretch release adhesive structure of patient structure 220 includes a backing layer 222 and adhesive layers 224 and 226 of the same or different pressure-sensitive adhesive compositions on opposite major surfaces of the backing layer 222. The depths or thicknesses of each of the layers illustrated in any of the Figures are not drawn to scale with respect to one another. In the embodiment of FIGS. 3-5, device structure 240 includes a second stretch release adhesive structure which (similar to the first stretch release adhesive structure) includes a backing layer 242 and adhesive layers 244 and 246 of the same or different pressure-sensitive adhesive compositions on opposite major surfaces of the backing layer 242. Prior to use of application system 200, the external or exposed surfaces of the adhesive layers of patient structure 220 and device structure 240, respectively, can be covered by the liners or protective layers 221 and 241, respectively (see FIG. 4) as known in the art. Liners or covers for use in the present invention can, for example, be formed from conventional or developed materials, such as a silicone release paper, for covering adhesives, which preferably can be easily removed to provide subsequent bonding of adhesive to an object.

Backing layers 222 and 242, can, for example, be formed from a stretchable polymeric foam layer as known in the art of stretch release adhesive systems. Backing layers 222 and 242 can alternatively be formed from a stretchable polymeric film layer as known in the art. The choice of polymeric foam or polymeric film can readily be determined depending on the desired performance properties of application system 200. Polymeric foams can, for example, provide increased conformability and resiliency properties for adherence to surfaces having surface irregularities. Polymeric films can increase load bearing strength and rupture strength of the structures. Combinations of foams and films can be used as well. Suitable polymeric foams and films and combinations thereof are discussed, for example, in U.S. Pat. Nos. 5,516,581 and 6,572,945.

Solid adhesives can be used as an alternative to stretchable backing layers having adhesive layers as described above. Stretchable solid adhesives which are generally elastic in nature and include rubber-based and acrylate-solid adhesives as know in the art. A solid adhesive inherently provide a backing layer as well as two adhesive layers as a unit. As known in the art, a solid adhesive layer can include sublayers or mixtures so that one of its adhesive surface is the same or different than its other adhesive surface.

Patient structures 220 and device structure 240 can, for example, include tabs 222a and 242a, respectively. As described above, tabs 222a and 242a facilitate the stretch release of each adhesive tape structure. Tabs 222a can be formed as extensions of backing layer 222, while tabs 242a can be formed as extensions of backing layer 242. Adhesive layers 224, 226, 244 and 246 can each cover a similar area of the backing layers 222 and 242, respectively.

In use, application system 200 is, for example, removed from a package. Sensor 300 or other medical device can be distributed with application system 200 or separately therefrom. If the medical device is distributed with application system 200, it can be attached to adhesive layer 246 or separate therefrom. Preferably, adhesive layers 246 and 224 are covered with a cover layer for protection as described above. After application system 200 is removed from its package, the protective cover layers are removed. Adhesive layer 224 of patient structure 220 is then adhered to the patient. Sensor 300 or other medical device is then adhered to adhesive layer 246 of device structure 240. A medical procedure using sensor 300 or other medical device is then performed.

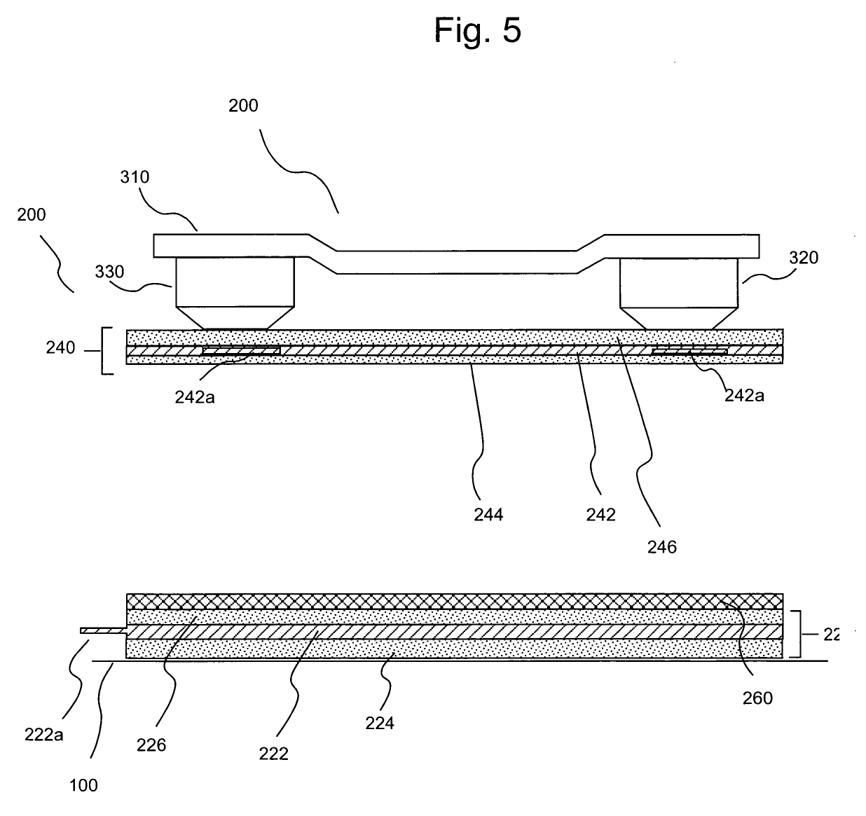

Following the procedure, device structure 240 and attached sensor 300 are removed from connection with intermediate layer 260 by pulling one or both of release tabs 242a to deform backing layer 242 and adhesive layer 244 and, thereby debond adhesive layer 244 from intermediate layer 260 (see FIG. 5). Intermediate layer 260 is preferably non-stretchable and can be formed from many polymeric and/or other materials as known in the art. Intermediate layer acts, in part, to shield patient structure 220 from stretching forces when device structure 240 is removed so that patient structure 220 remains in connection with the patient. Upon stretching of backing layer 242, adhesive layer 246 may also debond from sensor 300. Alternatively, sensor 300 can be removed therefrom in a later step. As discussed above, patient structure 220 preferably remains in connection with patient tissue 100 at this point and, as further described above, can act as a bandage or tissue covering until removal is desirable. When removal of patient structure is desired, one or both of tabs 22a can be pulled to stretch/deform backing layer 222 and adhesive layer 224 to debond adhesive layer 224 from patient tissue 100.

As illustrated, for example, in FIG. 3 when application system 200 is used in connection with a sensor for detection of extravasation (or certain other medical procedures), an open region 280 is preferably provided to allow observation and/or palpation of the area of an injection site X. Sensor configurations that provide such an area for palpation and/or observation are discussed, for example, in U.S. Pat. No. 6,408,204, assigned to the assignee of the present invention, the disclosure of which is incorporated herein by reference.

Although the present invention has been described in detail in connection with the above embodiments and/or examples, it should be understood that such detail is illustrative and not restrictive, and that those skilled in the art can make variations without departing from the invention. The scope of the invention is indicated by the following claims rather than by the foregoing description. All changes and variations that come within the meaning and range of equivalency of the claims are to be embraced within their scope.

1. A medical system for attachment to a patient, comprising:
a patient structure comprising a patient structure backing layer comprising a first major surface and a second major surface, a first patient structure adhesive layer covering at least a portion of the first major surface and being adapted to adhere to the patient; and
a device structure comprising a device structure backing layer comprising a third major surface and a fourth major surface, a first device structure adhesive layer covering at least a portion of the fourth major surface, the third major surface being in removable connection with the patient structure.

2. The system of claim 1 wherein the patient structure is adapted to remain adhered to the patient after the device backing layer is removed from connection with the patient structure.

3. The system of claim 2 wherein the patient structure forms at least part of a bandage system.

4. The system of claim 1 further comprising an intermediate layer; the patient structure further comprising a second patient structure adhesive layer covering at least a portion of the second major surface, the second patient structure adhesive being adhered to the intermediate layer; the device structure further comprising a second device structure adhesive layer covering at least a portion of the third major surface of the device structure backing layer, the second device structure adhesive layer being adhered to the intermediate layer.

5. The system of claim 4 wherein the device backing layer and the second device structure adhesive layer are stretchable together to effect debonding of the second device structure adhesive layer from the intermediate layer.

6. The system of claim 4 wherein the patient structure backing layer and the first patient structure adhesive layer are stretchable together to effect debonding of the first patient structure adhesive layer from the patient.

7. The system of claim 1 wherein further comprising a medical device attachable to the first device structure adhesive layer.

8. The system of claim 1 wherein 7 wherein the medical device is a sensor.

9. The system of claim 8 wherein the sensor is an extravasation sensor.

10. The system of claim 9 wherein the extravasation sensor comprises at least one transmitter for transmitting electromagnetic energy in the range of 300 MHz to 30 Ghz into tissue of the patient and at least one receiver to receive a resultant signal.

11. The system of claim 10 wherein the at least one transmitter is adapted to transmit electromagnetic energy having a frequency greater than 1.5 GHz.

12. A system for removable attachment of a medical device to a patient, comprising:
a patient structure comprising a first patient structure adhesive layer adapted to adhere to a patient and a cover layer in connection with the first patient structure adhesive layer;
a medical device structure in removable connection with the cover layer of the patient structure, the medical device structure being removable from attachment to the cover layer of the patient structure without removing the patient structure from attachment to the patient, the medical device structure including a first device structure adhesive layer on at least a portion of a surface opposite of a surface thereof that is in removable connection with the cover layer, the first device structure adhesive layer being adapted to attach the medical device to the medical device structure.

13. The system of claim 12 wherein the patient structure forms at least part of a bandage system.

14. The system of claim 12 further comprising a first removable protective layer in operative connection with the first patient structure adhesive layer, the first protective layer being removable to allow contact of the first patient structure adhesive layer with the patient.

15. The system of claim 12 further comprising a second removable protective layer in operative connection with the first device structure adhesive layer, the second protective layer being removable to allow contact of the first device structure adhesive layer with the medical device.

16. A method of removably attaching a medical device to a patient, comprising:
attaching an attachment system to the patient, the attachment system comprising a patient structure comprising a first adhesive layer adapted to adhere to the patient and a cover layer in connection with the first adhesive layer, the attachment system further comprising a medical device structure removably attached to the cover layer of the patient structure, the medical device structure comprising a device structure adhesive on a surface thereof opposite of a surface thereof in removable connection with the cover layer, the device structure adhesive being adapted to attach the medical device to the medical device structure.

17. The method of claim 16 further comprising removing the medical device structure from attachment to the cover layer of the patient structure without removing the patient structure from attachment to the patient.

18. The method of claim 16 further comprising the step of attaching the medical device to the device structure adhesive.

19. The method of claim 18 wherein the medical device is an extravasation sensor.

20. The method of claim 19 wherein the extravasation sensor comprises at least one transmitter for transmitting electromagnetic energy in the range of 300 MHz to 10 Ghz into tissue of the patient and at least one receiver to receive a resultant signal.
